(19) United States
(12) Patent Application Publication
(10) Pub. No.: US 2007/0041588 A1
(43) Pub. Date: Feb. 22, 2007

(54) Motor operation noise elimination circuit of portable multimedia player

(76) Inventor: Cheng-Li Lin, Taipei City (TW)

Correspondence Address: HARNESS, DICKEY & PIERCE, P.L.C., P.O. BOX 8910, RESTON, VA, 20195, US

(21) Appl. No.: 11/204,992

(22) Filed: Aug. 17, 2005

(51) Int. Cl. A61F 11/06; G10K 11/16; H03B 29/00

(52) U.S. Cl. 381/71.1

(57) ABSTRACT

A motor operation noise elimination circuit of a portable multimedia player is provided, in which a first sound sensor is utilized to sense a first sound signal generated by motor operation during recording and a second sound signal input in the portable multimedia player for recording. Then the first sound signal sensed by the second sound sensor is phase-reversed in the noise elimination unit and is combined into the first and second sound signals sensed by the first sound sensor during recording, comprising: a second sound sensor, used to sense a first sound signal generated by the motor, and a noise elimination unit, used to receive the first and second sound signals sensed by the first sound sensor, and the first sound signal sensed by the second sound sensor, then a combination operation is executed, thus eliminating the first sound signal and outputting the second sound signal.

### BACKGROUND

1. Field of the Invention

The invention relates to a noise elimination circuit, and in particular to a motor operation noise elimination circuit of a portable multimedia player (PMP).

2. Related Art

With the advent of the multimedia era, nowadays users of multimedia devices are able to have the benefit of pleasure and enjoyment offered by portable multimedia players at any time and any place of their own choice. For the portable multimedia player, the multimedia data file is stored in a miniature hard disk. However, during operation of the hard disk drive, the vibration caused by the driving motor will induce a noise problem. Thus in the program recording process of PMP, the noise caused by vibration of the motor is recorded together with the sound desired to be recorded, as such reducing the sound recording quality of the portable multimedia player.

Refer to Patent Publication No. 446258 of the Republic of China, wherein is disclosed an earphone, capable of eliminating environment noise, in which a microphone disposed in the vicinity of the trumpet of an earphone is used to absorb the noise of the nearby environment (for example, ear cavity), and a sound wave of reverse phase (180°) of the noise produced by a circuit is used to cancel and eliminate the original noise, thus achieving the purpose of pressure balance and noise elimination in a specific environment.

Moreover, refer to Patent Publication No. 535967, wherein is disclosed a mobile phone communication noise elimination device used in a vehicle, including a sensor placed near the source of noise in the vehicle, an analog/digital signal converter electrically connected to the sensor, a digital signal processor electrically connected to the analog/digital signal converter, a digital/analog signal converter electrically connected to the digital signal processor, and an audio signal generation unit electrically connected to the digital/analog signal converter. Upon receiving the analog audio signal transmitted from the digital/analog signal converter, the audio signal generation unit sends out an audio signal having an equal magnitude, but reverse phase relative to the noise. In this way, the audio signal thus produced is used to cancel out and eliminate the noise, thus improving sound receiving quality of a mobile phone in communication.

Though the afore-mentioned noise elimination circuit can be used to improve the sound receiving quality of mobile phone, yet the prices of digital operators, compensation circuits, digital signal processors, analog/digital signal converters and digital/analog signal converters are pretty high, thus raising the cost of noise elimination circuits.

Therefore, the research and development of a low cost motor operation noise elimination circuit of a portable multimedia player having improved sound recording quality is one of the most important tasks in this field.

### SUMMARY OF THE INVENTION

In view of the above-mentioned problems and shortcomings of the prior art, the object of the invention is to provide a motor operation noise elimination circuit of a portable multimedia player (PMP), which is utilized to eliminate or cancel out the noise of motor operation during a sounding recording process, by making use of the noise signal of motor operation, so as to avoid the appearance of noise caused by motor operation in the sound recorded, thus raising the sound recording quality of the portable multimedia player. Therefore, to achieve the above-mentioned objective, the invention provides a motor operation noise elimination circuit of a portable multimedia player, wherein a first sound sensor is utilized to sense a first sound signal, generated by motor operation and a second sound signal input to the portable multimedia player for recording, then the first sound signal sensed by the second sound sensor is phase-reversed in the noise elimination unit and is combined into the first and second sound signals' sensed by the first sound sensor during recording, thus achieving the objective of eliminating the first sound signal (namely, the noise) and outputting the second sound signal (namely, the sound desired to be recorded), including a second sound sensor and a noise elimination unit:

A second sound sensor, used to sense a first sound signal generated by the motor operation, and a noise elimination unit, used to receive the first and second sound signals sensed by the first sound sensor, and the first sound signal sensed by the second sound sensor, then execute a combination operation to eliminate the first sound signal and output the second sound signal.

Furthermore, a sound recording unit is provided in the circuit of the invention to record the second sound signal output by the noise elimination unit. Through the application of the motor operation noise elimination circuit of the portable multimedia player of the invention, the input sound to be recorded and noise generated by motor operation are obtained through two sound sensors, such, that the noise that is mixed with input sound to be recorded as sensed by a sound sensor is eliminated and cancelled out by the noise sensed by the other sound sensor. As such, through the circuit design of the invention, the noise generated by motor operation can be effectively eliminated from the output of the circuit, thereby raising the sound recording quality of the portable multimedia player.

Further scope of the applicability of the invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The invention will become more fully understood from the detailed description given below, which is for illustration only and thus is not limitative of the invention:

FIG. 1 is a structure diagram of a motor operation noise elimination circuit of a portable multimedia player according to an embodiment of the invention;

FIG. 2A is a circuit diagram of a differential amplifier used in a motor operation noise elimination circuit of a portable multimedia player according to an embodiment of the invention;

FIG. 2B is a circuit diagram of a similar differential amplifier used in a motor operation noise elimination circuit of a portable multimedia player according to another embodiment of the invention; and

FIG. 3 is a cross section diagram of a sound sensor portion of a portable multimedia player according to an embodiment of the invention.

### DETAILED DESCRIPTION OF THE INVENTION

The purpose, construction, features, and functions of the invention can be appreciated and understood more thoroughly through the following detailed description with reference to the attached drawings.

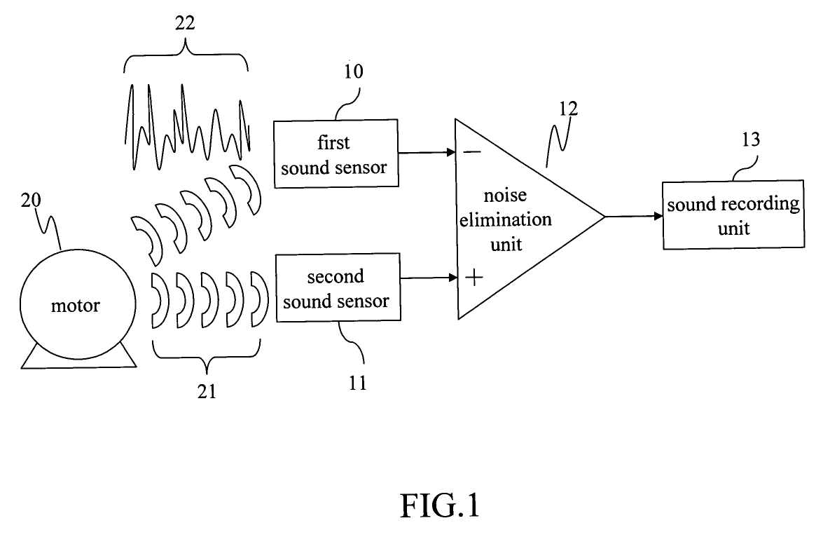

Refer to FIG. 1 for a structure diagram of a motor operation noise elimination circuit of a portable multimedia player according to an embodiment of the invention, including: a first sound sensor 10, a second sound sensor 11, a noise elimination unit 12, a motor 20 and a sound recording unit 13. Each of the above-mentioned devices is described in detail as follows.

A first sound sensor 10 is provided in a portable multimedia player, also a plurality of sound receiving holes are provided on the outer shell of a portable multimedia player, so that the first sound signal 21 generated by the motor 20 and the second sound signal 22 (for example, music or voice) input from the outside environment can be sensed by the first sound sensor 10. During the program recording process, the first sound signal 21 sensed by the second sound sensor 11 is combined into the first sound signal 21 and the second sound signal 22, sensed by the first sound sensor 10. The first sound sensor 10 may be composed of a capacitance microphone, a movable-coil microphone or a silicon crystal microphone.

A second sound sensor 11 is provided in a portable multimedia player, which is utilized to sense the vibration noise (namely, the first sound signal 21) generated by the motor 20 during its operation, wherein the second sound sensor 11 may be composed of a capacitance microphone, a movable-coil microphone or a silicon crystal microphone.

A noise elimination unit 12, connected respectively to a first sound sensor 10 and a second sound sensor 11 for receiving a first sound signal 21 and a second sound signal 22, utilizes each of them to perform a combination operation, so as to eliminate the first sound signal 21 and output the second sound signal 22. The noise elimination unit 12 can be composed of a differential amplifier having two input terminals and an output terminal. The first sound sensor 10 is connected to a reverse phase input terminal of a differential amplifier, and the second sound sensor 11 is connected to a positive phase input terminal of a differential amplifier.

A sound recording unit 13, connected to a noise elimination unit 12, is used to receive the second sound signal 22 outputs by the noise elimination unit 12. The sound recording unit 13 can be made of a miniature hard disk or a flash memory.

A motor 20 is disposed in a portable multimedia player, and used to provide the power required for driving the storage medium (for example, miniature hard disk drive).

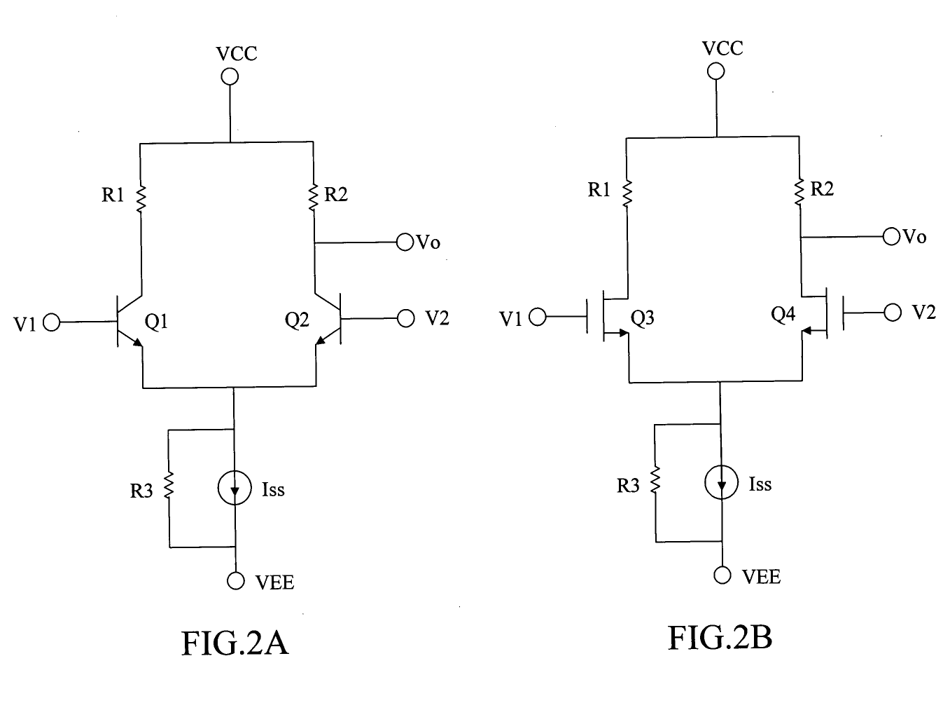

Subsequently, refer to FIG. 2A, which shows a circuit diagram of a differential amplifier used in a motor operation noise elimination circuit of a portable multimedia player according to an embodiment of the invention, including: a resistor R1, a resistor R2, a resistor R3, a transistor Q1, a transistor Q2, and a current source Iss.

Firstly, a first sound signal 21 and a second, sound signal 22 sensed by the first sound sensor 10 is input in an input terminal V1 of a differential amplifier, and the input terminal V1 is connected to a base of transistor Q1. Then, a first sound signal 21 sensed by a second sound sensor 11 is input in an input terminal V2 of a differential amplifier, the input terminal V2 is connected to a base of transistor Q2, a collector of transistor Q1 is connected to one end of resistor R1, the other end of resistor R1 is connected to a positive voltage supply VCC, an emitter of transistor Q1 is connected to one end of the current source Iss, the other end of the current source Iss is connected to a negative voltage supply, resistor R3 is connected in parallel with the current source Iss, a collector of transistor Q2 is connected to one end of resistor R2, the other end of resistor R2 is connected to the positive voltage supply VCC, an emitter of transistor Q2 is connected to one end of resistor R2, the other end of resistor R2 is connected to the positive voltage supply VCC, an emitter of transistor Q2 is connected to one end of the current source Iss, and the other end of the current source Iss is connected to the negative voltage supply.

Upon executing the combination operation of a differential amplifier, the second sound signal 22 is output from an output terminal Vo, which is connected to a sound recording unit 13 to record the second sound signal 22. In addition, refer to FIG. 2B, which shows a circuit diagram of a similar differential amplifier used in a motor operation noise elimination circuit of a portable multimedia player according to another embodiment of the invention, the difference with FIG. 2A is that transistor Q1 and Q2 in FIG. 2A are replaced by MOSFET Q3 and MOSFET Q4 in FIG. 2B, the other elements shown in the two drawings are similar, which will not be repeated here for brevity's sake.

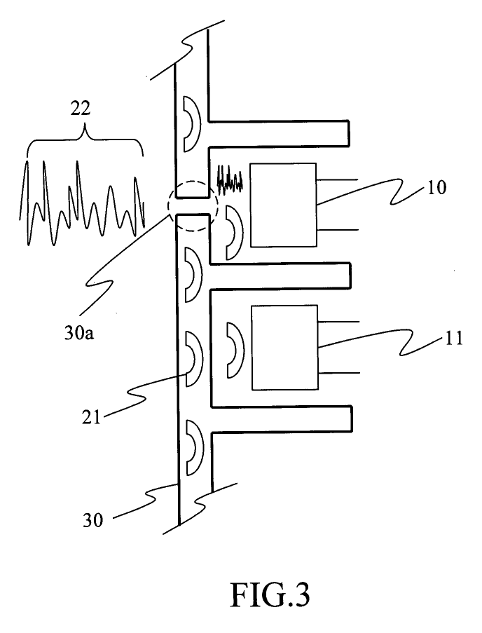

Finally, refer to FIG. 3 for a cross section diagram of a sound sensor portion of a portable multimedia player according to an embodiment of the invention, wherein the first sound sensor 10 is disposed nearby the second sound sensor 11, and a sound receiving hole 30a is provided on a shell 30 to enable the first sound sensor 10 to sense the second sound signal 22 input from the outside environment. In the sound recording process, the vibration of the motor 20 operation causes the shell 30 to vibrate, thus generating the first sound signal 21. The second sound sensor 11 is used to sense only the first sound signal 21, thus there is no sound receiving hole 30a provided on the shell 30 near the position of the second sound sensor 11, so that it only senses the first sound signal 21 caused by the vibration of the motor 20 and shell 30.

Through the application of the motor operation noise elimination circuit of the portable multimedia player of the invention, the input sound to be recorded and noise generated by motor operation are obtained through two sound sensors, such, that the noise that is mixed with input sound to be recorded as sensed by a sound sensor is eliminated and cancelled out by the noise sensed by the other sound sensor. As such, through the circuit design of the invention, the noise generated by motor operation can be effectively eliminated from the output of the circuit, thereby raising the sound recording quality of the portable multimedia player.

Knowing the invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

What is claimed is:

1. A motor operation noise elimination circuit of a portable multimedia player, in which a first sound sensor is utilized to sense a first sound signal generated by motor operation during recording and a second sound signal input to the portable multimedia player for recording, and then the first sound signal sensed by the second sound sensor is phase-reversed in the noise elimination unit and is combined into the first and second sound signals sensed by the first sound sensor during recording, thus eliminating the first sound signal, comprising:
a second sound sensor, used to sense a first sound signal generated by motor operation; and
a noise elimination unit, used to receive the first and second sound signals sensed by the first sound sensor, and the first sound signal sensed by the second sound sensor, then execute a combination operation to eliminate the first sound signal and output the second sound signal.

2. The motor operation noise elimination circuit of a portable multimedia player as claimed in claim 1, wherein the first sound sensor is a capacitance microphone.

3. The motor operation noise elimination circuit of a portable multimedia player as claimed in claim 1, wherein the first sound sensor is a movable-coil microphone.

4. The motor operation noise elimination circuit of a portable multimedia player as claimed in claim 1, wherein the first sound sensor is a silicon crystal microphone.

5. The motor operation noise elimination circuit of a portable multimedia player as claimed in claim 1, wherein the second sound sensor is a capacitance microphone.

6. The motor operation noise elimination circuit of a portable multimedia player as claimed in claim 1, wherein the second sound sensor is a movable-coil microphone.

7. The motor operation noise elimination circuit of a portable multimedia player as claimed in claim 1, wherein the second sound sensor is a silicon crystal microphone.

8. The motor operation noise elimination circuit of a portable multimedia player as claimed in claim 1, wherein the noise elimination unit is a differential amplifier.

9. The motor operation noise elimination circuit of a portable multimedia player as claimed in claim 8, wherein the first sound signal sensor is connected to a reverse phase input terminal of the differential amplifier.

10. The motor operation noise elimination circuit of a portable multimedia player as claimed in claim 8, wherein the first sound signal sensor is connected to a positive phase input terminal of the differential amplifier.

11. The motor operation noise elimination circuit of a portable multimedia player as claimed in claim 1, further comprising:
a sound recording unit, which is used to record the second sound signal output by the noise elimination unit.
